(19) United States
(12) Patent Application Publication
(10) Pub. No.: US 2026/0219250 A1
(43) Pub. Date: Jul. 30, 2026

(54) FARM FIELD MANAGEMENT APPARATUS, FARM FIELD MANAGEMENT METHOD, AND NON-TRANSITORY COMPUTER READABLE MEDIUM

(71) Applicant: Yokogawa Electric Corporation, Tokyo (JP)

(72) Inventors: Emiko HATSUGAI, Tokyo (JP); Junshan WANG, Tokyo (JP); Hirari SATOU, Tokyo (JP)

(21) Appl. No.: 19/148,672

(22) PCT Filed: Mar. 5, 2024
(86) PCT No.: PCT/JP2024/008376

§ 371 Date: Jul. 16, 2025

(30) Foreign Application Priority Data
Mar. 23, 2023 (JP) 2023-046960

(51) Int. Cl. G01N 33/00; A01G 7/02; A01G 7/04; A01G 25/16; G05D 1/689; G05D 105/80; G05D 107/20

(52) U.S. Cl. CPC G01N 33/0098; A01G 7/02; A01G 7/045; A01G 25/167; G05D 1/689; G05D 2105/80; G05D 2107/21

(57) ABSTRACT

A farm field management apparatus may include an acquisition unit which acquires a first measured value at a first point in a farm field which is measured by a first sensor provided at the first point, and a second measured value at a second point in the farm field which is measured by a second sensor provided at the second point, a determination unit which determines whether to cause a mobile robot to which a third sensor is mounted to move to a third point between the first point and the second point and to cause the mobile robot to measure a third measured value at the third point by the third sensor, and an instruction unit which instructs, when the determination unit determines that the third measured value at the third point is to be measured, the mobile robot to perform measurement at the third point.

The contents of the following patent application(s) are incorporated herein by reference:
NO. 2023-046960 filed in JP on Mar. 23, 2023.

### BACKGROUND

### 1. Technical Field

The present invention relates to a farm field management apparatus, a farm field management method, and a program.

### 2. Related Art

Patent Document 1 discloses a leaf surface environment sensor which detects an illuminance (or a degree of sunlight) on a front surface of a leaf or a temperature and a humidity (one of a temperature or a humidity or both) on a back side of the leaf, a leaf color, a concentration of carbon dioxide emitted from the leaf, or the like.

### RELATED ART DOCUMENTS

### Patent Documents

Patent Document 1: Japanese Patent Application Publication No. 2022-100732

### GENERAL DISCLOSURE

A farm field management apparatus according to an aspect of the present invention may include an acquisition unit which acquires a first measured value at a first point in a farm field which is measured by a first sensor provided at the first point, and a second measured value at a second point in the farm field which is measured by a second sensor provided at the second point. The farm field management apparatus may include a determination unit which determines whether to cause a mobile robot to which a third sensor is mounted to move to a third point between the first point and the second point based on the first measured value and the second measured value and to cause the mobile robot to measure a third measured value at the third point by the third sensor. The farm field management apparatus may include an instruction unit which instructs, when the determination unit determines that the third measured value at the third point is to be measured, the mobile robot to perform measurement at the third point.

In the farm field management apparatus, when a difference between the first measured value and the second measured value is equal to or greater than a threshold, the determination unit may determine that the mobile robot is to be caused to measure the third measured value at the third point by the third sensor.

Any of the farm field management apparatuses may further include an estimation unit which estimates, when a difference between the first measured value and the second measured value is less than a threshold, the third measured value at the third point by interpolation between the first measured value and the second measured value.

In any of the farm field management apparatuses, when a difference between measured values in a same time slot of the first measured value or the second measured value is equal to or greater than a threshold, the determination unit may determine that the mobile robot is to be caused to measure the third measured value at the third point by the third sensor.

Any of the farm field management apparatuses may further include an estimation unit which estimates, when a difference between measured values in a same time slot of the first measured value or the second measured value is less than a threshold, the third measured value at the third point by interpolation between the first measured value and the second measured value.

In any of the farm field management apparatuses, the estimation unit may perform the interpolation by using a trained predictive model in which temperatures, humidities, and light quantities at the first point and the second point which are respectively measured by the first sensor and the second sensor are set as explanatory variables, and a temperature, a humidity, and a light quantity at the third point which are measured by the third sensor unit are set as objective variables.

In any of the farm field management apparatuses, the instruction unit may instruct the mobile robot to perform the measurement at the third point in a manner that as the difference between the first measured value and the second measured value becomes larger, a frequency to measure the third measured value at the third point by the third sensor is increased.

In any of the farm field management apparatuses, the third point may include a plurality of third points, and when a difference between the first measured value and the second measured value is equal to or greater than a threshold, the determination unit may determine that the mobile robot is to be caused to measure the third measured value at each of the plurality of third points by the third sensor.

The instruction unit may instruct the mobile robot to perform the measurement at the third point in a manner that as the difference between the first measured value and the second measured value becomes larger, a number of the third points at which the mobile robot is instructed to perform the measurement is increased.

In any of the farm field management apparatuses, the instruction unit may decide a position of the third point based on the first measured value and the second measured value.

Any of the farm field management apparatuses may further include a generation unit which generates a measured value distribution of the farm field based on the first measured value, the second measured value, and the third measured value.

In any of the farm field management apparatuses, the measured value distribution may include at least one distribution of a temperature, a humidity, an electrical conductivity, or a hydrogen ion index of a root part of a plant, or a front surface temperature, an ambient temperature, a humidity, a light quantity, or a carbon dioxide concentration of a stem, leave, and fruit part of the plant.

In any of the farm field management apparatuses, the instruction unit may instruct environmental control equipment in the farm field to adjust at least one of a moisture content, an electrical conductivity, or a hydrogen ion index of a soil in the farm field or a temperature, a humidity, a light quantity, or a carbon dioxide concentration in the farm field based on a predictive model representing a relationship between the measured value distribution and an occurrence status of a defect of the plant or a harvest result of the plant such that the plant reaches a predetermined growing condition.

In any of the farm field management apparatuses, the instruction unit may identify the growing condition of the plant based on three-dimensional position information of the plant which is obtained by a detection result by an optical sensor existing in the farm field.

In any of the farm field management apparatuses, the optical sensor may be mounted to the mobile robot.

A farm field management method according to an aspect of the present invention may include acquiring a first measured value at a first point in a farm field which is measured by a first sensor provided at the first point, and a second measured value at a second point in the farm field which is measured by a second sensor provided at the second point. The farm field management method may include determining whether to cause a mobile robot to which a third sensor is mounted to move to a third point between the first point and the second point based on the first measured value and the second measured value and to cause the mobile robot to measure a third measured value at the third point by the third sensor. The farm field management method may include instructing, when it is determined in the determining that the third measured value at the third point is to be measured, the mobile robot to perform measurement at the third point.

A program according to an aspect of the present invention may cause a computer to function as an acquisition unit which acquires a first measured value at a first point in a farm field which is measured by a first sensor provided at the first point, and a second measured value at a second point in the farm field which is measured by a second sensor provided at the second point. The program according to an aspect of the present invention may cause the computer to function as a determination unit which determines whether to cause a mobile robot to which a third sensor is mounted to move to a third point between the first point and the second point based on the first measured value and the second measured value and to cause the mobile robot to measure a third measured value at the third point by the third sensor. The program according to an aspect of the present invention may cause the computer to function as an instruction unit which instructs, when the determination unit determines that the third measured value at the third point is to be measured, the mobile robot to perform measurement at the third point.

The summary clause does not necessarily describe all necessary features of the embodiments of the present invention. The present invention may also be a sub-combination of the features described above.

### BRIEF DESCRIPTION OF THE DRAWINGS

FIG. 1 illustrates a situation in which a mobile robot moves in a farm field for cultivating plants.

FIG. 2 illustrates an example of an overall configuration of a farm field management system according to the present embodiment.

FIG. 3 illustrates an example of a cultivation rack.

FIG. 4 illustrates an example of a functional block of a farm field management apparatus.

FIG. 5 is a flowchart illustrating an example of a procedure to determine whether a measured value between points is acquired by an estimation or is acquired by a measurement by using the mobile robot.

FIG. 6 illustrates an example of a hardware configuration.

### DESCRIPTION OF EXEMPLARY EMBODIMENTS

Hereinafter, the present invention will be described through embodiments of the invention, but the following embodiments do not limit the invention according to claims. In addition, not all of the combinations of features described in the embodiments are essential to the solving means of the invention.

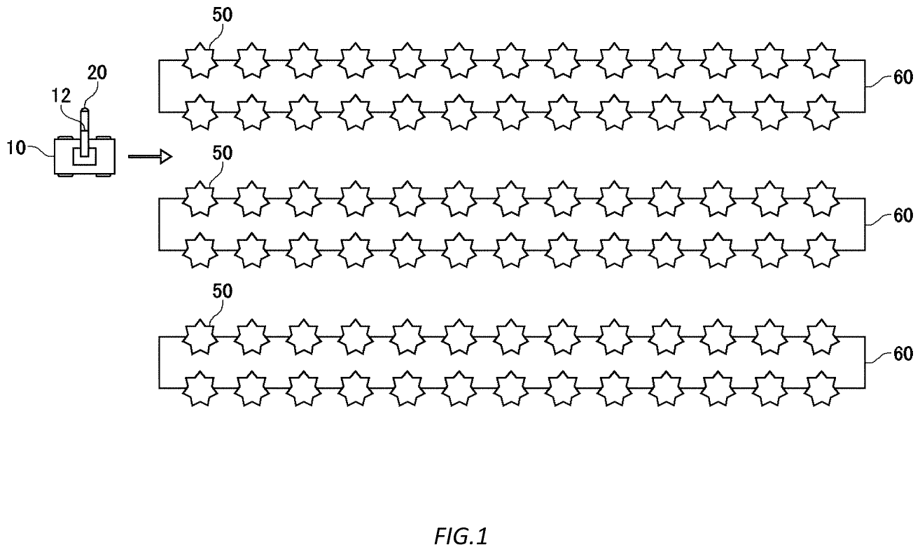

FIG. 1 illustrates a situation in which a mobile robot 10 moves in a farm field for cultivating a plant 50 such as a vegetable or fruit. The mobile robot 10 is a vehicle moving on a ground. The mobile robot 10 may be a flight vehicle such as an unmanned aircraft moving in the air, or a ship moving on water. The mobile robot 10 may move between cultivation racks 60 for cultivating the plant 50.

In the present embodiment, the farm field is an artificial light powered plant factory for cultivating the plant 50 by using artificial light such as an LED or an incandescent lamp as a light source. However, the farm field may be a solar powered plant factory for cultivating the plant 50 by using sunlight as the light source.

The mobile robot 10 includes an arm 12 and a sensor unit 20 provided at a distal end of the arm 12. The arm 12 may be an articulated arm unit rotatably provided to a main body of the mobile robot 10. The sensor unit 20 includes various types of sensors which gauge an environmental condition in a surrounding of the plant 50 and a growing condition of the plant 50. The sensor unit 20 includes various types of sensors which respectively measure a temperature, a humidity, a light quantity, and a carbon dioxide concentration of a stem, leave, and fruit part of the plant 50. The sensor unit 20 may include an optical sensor for gauging the growing condition of the plant 50. The optical sensor may be a camera or a laser scanner. The camera may be a twin-lens 3D camera or a time of flight (ToF) camera. Three-dimensional position information of the plant 50 may be generated by using a detection result of the optical sensor.

Various types of sensors which measure a temperature, a humidity (moisture content), an electrical conductivity (EC), and a hydrogen ion index (pH) of a root part (culture medium part) of the plant 50, and various types of sensors which respectively measure a front surface temperature, an ambient temperature, a humidity, a light quantity, and a carbon dioxide concentration of the stem, leave, and fruit part of the plant 50 are also installed in the cultivation rack 60.

In order to precisely grasp the environmental condition of the individual plant 50 and the growing condition of the plant 50, the various types of sensors are preferably provided to the individual plant 50. However, when the various types of sensors are provided to the individual plant 50, cost is increased. In addition, when data is transmitted from a large number of sensors, communication is strained, and burden on an apparatus which processes the data may also be increased.

Therefore, in a farm field management system according to the present embodiment, while a number of sensors provided in the farm field is suppressed, decrease in the precision to grasp the environmental condition of the surrounding of the individual plant 50 and the growing condition of the plant 50 is suppressed.

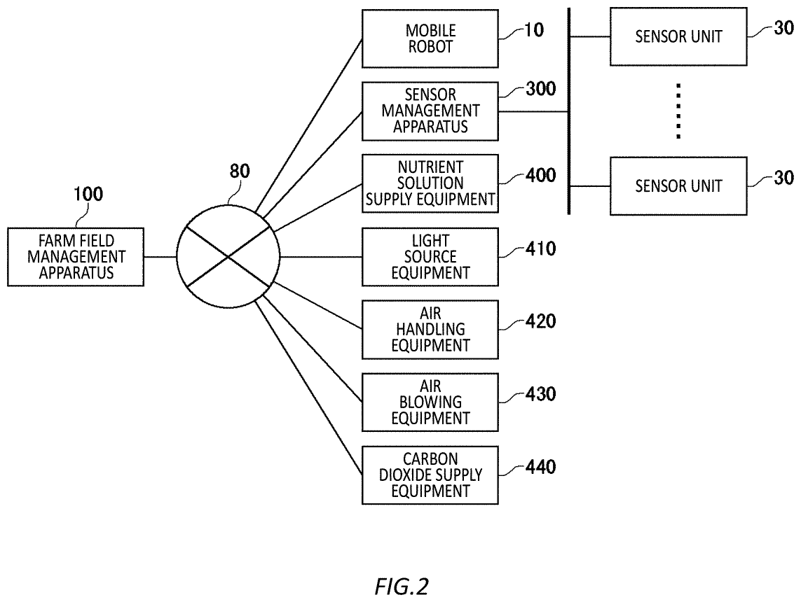

FIG. 2 illustrates an example of an overall configuration of the farm field management system according to the present embodiment. The farm field management system includes a farm field management apparatus 100, the mobile robot 10, a plurality of sensor units 30, a sensor management apparatus 300, nutrient solution supply equipment 400, light source equipment 410, air handling equipment 420, air blowing equipment 430, and carbon dioxide supply equipment 440. The nutrient solution supply equipment 400, the light source equipment 410, the air handling equipment 420, the air blowing equipment 430, and the carbon dioxide supply equipment 440 are examples of environmental control equipment. Each of a plurality of mobile robots 10 may move in a different or same area in the farm field.

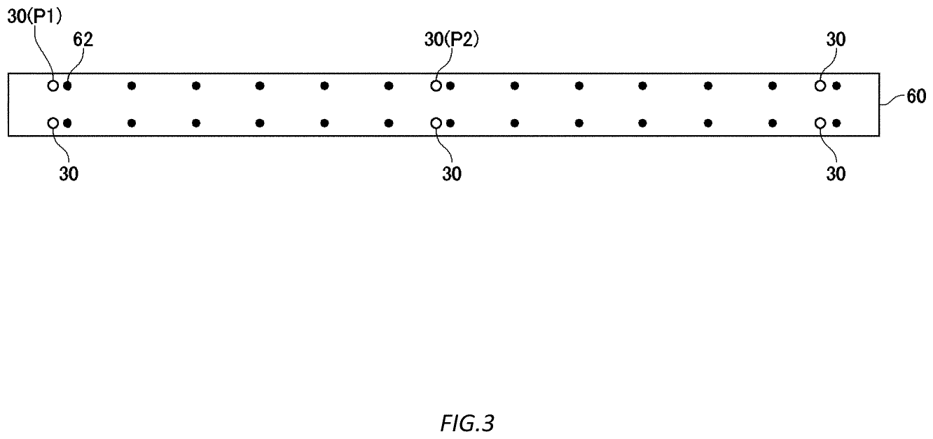

FIG. 3 illustrates an example of installation locations of the sensor units 30. The sensor unit 30 is provided in a surrounding of several planting pots 62 among planting pots 62 of all the plants 50 included in the cultivation rack 60. The sensor unit 30 may be provided to be equally spaced for each set of multiple planting pots 62. That is, the sensor unit 30 is not provided to each of all the planting pots 62 in the cultivation rack 60. A part of the sensor unit 30 may be provided to a stem, a leaf, or the like of the plant 50. A part of the sensor unit 30 may be provided in a culture medium.

The sensor unit 30 includes various types of sensors which measure a temperature, a humidity (moisture content), an electrical conductivity, and a hydrogen ion index of the root part (culture medium part) of the plant 50, and various types of sensors which respectively measure a temperature, a humidity, a light quantity, and a carbon dioxide concentration of the stem, leave, and fruit part of the plant 50. The sensor unit 30 may periodically perform measurement at a predetermined time interval.

The farm field management apparatus 100 manages various types of apparatuses in the plant factory. The farm field management apparatus 100 is connected to the mobile robot 10, the sensor management apparatus 300, the nutrient solution supply equipment 400, the light source equipment 410, the air handling equipment 420, the air blowing equipment 430, and the carbon dioxide supply equipment 440 via a network 80 to communicate with each other. The farm field management apparatus 100 may be connected to the plurality of sensor units 30 via the network 80 to communicate with each other.

The farm field management apparatus 100 and the sensor management apparatus 300 may be a computer having a central processing unit (CPU) and a memory. The nutrient solution supply equipment 400, the light source equipment 410, the air handling equipment 420, the air blowing equipment 430, and the carbon dioxide supply equipment 440 may be a computer having a central processing unit (CPU) and a memory.

The computer may be a computer such as a personal computer, a tablet type computer, a smartphone, a workstation, a server computer, or a general purpose computer, or may be a computer system in which a plurality of computers is connected to each other. Such a computer system is also a computer in a broad sense. The computer may be a dedicated computer desired to control an environment of the plant factory, or may be dedicated hardware achieved by a dedicated circuit. The computer may be implemented by a virtual computer environment. When the computer is used, the farm field management apparatus 100, the sensor management apparatus 300, the nutrient solution supply equipment 400, the light source equipment 410, the air handling equipment 420, the air blowing equipment 430, and the carbon dioxide supply equipment 440 are achieved when a program is executed by the computer.

The farm field management apparatus 100 controls the environment in the plant factory according to the growing condition of the plant 50 by controlling the mobile robot 10, the sensor management apparatus 300, the nutrient solution supply equipment 400, the light source equipment 410, the air handling equipment 420, the air blowing equipment 430, and the carbon dioxide supply equipment 440.

The sensor management apparatus 300 collects various types of measured values from each of the plurality of sensor units 30, and provides the measured values to the farm field management apparatus 100. The farm field management apparatus 100 may include the sensor management apparatus 300.

The nutrient solution supply equipment 400 supplies a nutrient solution containing each fertilizer component such as potassium or calcium to the cultivation rack 60 via a pump. The nutrient solution supply equipment 400 may adjust a fertilizer concentration and amount of the nutrient solution according to an instruction from the farm field management apparatus 100.

The light source equipment 410 includes a light source which emits artificial light such as the LED or the incandescent lamp provided to the cultivation rack 60, and illuminates the plant 50 with the artificial light from the light source. The light source equipment 410 may control a light quantity and an illumination period of the light source according to an instruction from the farm field management apparatus 100. When the farm field is a solar powered plant factory, the farm field management system may include, instead of the light source equipment 410, solar radiation amount control equipment which controls opening and closing of a curtain installed for a window or the like in order to adjust a radiation amount of sunlight with which the plant 50 is illuminated.

The air handling equipment 420 performs temperature and humidity conditioning on air in an indoor space of the plant factory, and causes the temperature and humidity conditioned air to circulate in the indoor space. The air handling equipment 420 may control a temperature and a humidity in the indoor space according to an instruction from the farm field management apparatus 100.

The air blowing equipment 430 includes a circulator or a fan which supplies wind to the indoor space of the plant factory. The air blowing equipment 430 may control an amount and an orientation of the wind supplied to the indoor space according to an instruction from the farm field management apparatus 100.

The carbon dioxide supply equipment 440 supplies carbon dioxide to the inside of the plant factory from a carbon dioxide tank. The carbon dioxide supply equipment 440 may control an amount of carbon dioxide to be supplied to the indoor space according to an instruction from the farm field management apparatus 100.

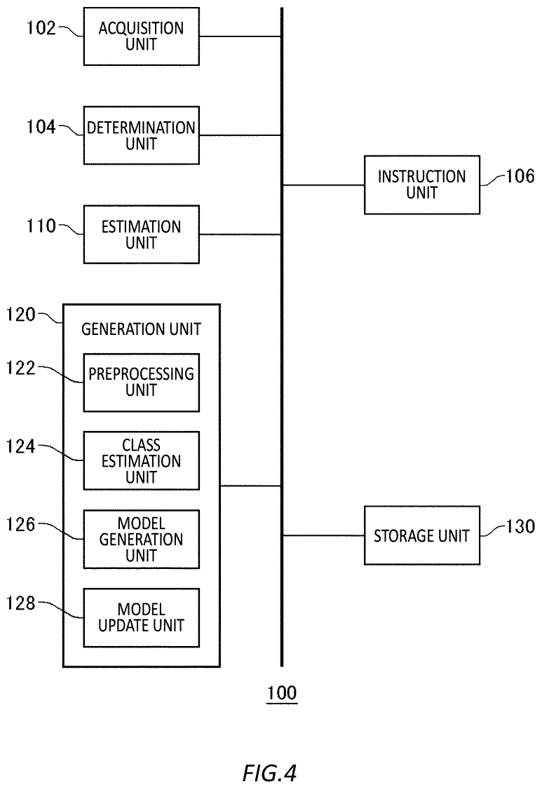

FIG. 4 is an example of a functional block of the farm field management apparatus 100. The farm field management apparatus 100 includes an acquisition unit 102, a determination unit 104, an instruction unit 106, an estimation unit 110, a generation unit 120, and a storage unit 130. The CPU included in the farm field management apparatus 100 may function as the acquisition unit 102, the determination unit 104, the instruction unit 106, the estimation unit 110, and the generation unit 120.

The acquisition unit 102 acquires each of measured values measured by each of the sensor units 30 provided in the farm field (plant factory). The acquisition unit 102 may periodically acquire each of the measured values measured by each of the sensor units 30 provided in the farm field at a predetermined interval. The acquisition unit 102 causes each of the measured values to be accumulated in the storage unit 130. For example, the acquisition unit 102 acquires a measured value M1 at a point P1 which is measured by the sensor unit 30 (P1) provided at the point P1 in the farm field, and a measured value M2 at a point P2 which is measured by the sensor unit 30 (P2) provided at the point P2 in the farm field.

As illustrated in FIG. 3, the point P1 and the point P2 are at positions separated by sandwiching a plurality of planting pots 62. That is, at least one plant 50 that is not a gauging target by the sensor unit 30 exists between the sensor units 30.

The determination unit 104 causes the mobile robot 10 to which the sensor unit 20 is mounted to move to the point P3 between the point P1 and the point P2 based on the measured value M1 and the measured value M2, and determines whether the mobile robot 10 is caused to measure a measured value M3 at the point P3 by the sensor unit 20.

When a difference between the measured value M1 and the measured value M2 is less than a threshold, the estimation unit 110 estimates the measured value M3 at the point P3 by interpolation between the measured value M1 and the measured value M2. When the difference between the measured value M1 and the measured value M2 is less than the threshold, the estimation unit 110 may estimate measured values at a plurality of points including the point P3 by the interpolation between the measured value M1 and the measured value M2. On the other hand, when the difference between the measured value M1 and the measured value M2 is equal to or greater than the threshold, there is a chance that the estimation unit 110 cannot precisely estimate the measured value M3 at the point P3 between the measured value M1 and the measured value M2. Thus, the determination unit 104 may determine that the mobile robot 10 is caused to measure the measured value M3 at the point P3 by the sensor unit 20.

When the difference between the measured value M1 and the measured value M2 is equal to or greater than the threshold, the instruction unit 106 may instruct the mobile robot 10 to perform the measurement at a plurality of points between the point P1 and the point P2. The instruction unit 106 may instruct the mobile robot 10 to perform the measurement at the point P3 such that, as the difference between the measured value M1 and the measured value M2 becomes larger, a frequency to measure the measured value M3 at the point P3 by the sensor unit 20 is increased. The instruction unit 106 may instruct the mobile robot 10 to perform the measurement at the point P3 at a first frequency per unit time period (for example, one day, one hour, or the like) when the difference between the measured value M1 and the measured value M2 is in a range from a first threshold to a second threshold, and the instruction unit 106 may instruct the mobile robot 10 to perform the measurement at the point P3 at a second frequency that is higher than the first frequency per unit time period (for example, one day, one hour, or the like) when the difference between the measured value M1 and the measured value M2 is equal to or greater than the second threshold. The instruction unit 106 may instruct the mobile robot 10 to perform the measurement at a plurality of points between the point P1 and the point P2 such that, as the difference between the measured value M1 and the measured value M2 becomes larger, a number of points to be measured by the sensor unit 20 between the point P1 and the point P2 is increased.

The instruction unit 106 instructs the mobile robot 10 to perform the measurement at the point P3 via the network 80. The instruction unit 106 may instruct the mobile robot 10 to perform the measurement by setting a way point between the point P1 and the point P2 as the point P3.

The instruction unit 106 may decide a position of the point P3 based on the measured value M1 and the measured value M2. When either the measured value M1 or the measured value M2 is out of a predetermined measured value range, the instruction unit 106 may determine the measured value as an abnormal value, and determine, as the point P3, a point closer to a point where the abnormal value is determined than the way point between the point P1 and the point P2. When either the measured value M1 or the measured value M2 differs by a predetermined percentage or more from an average value of measured values up to the previous measurement in a same time slot, the instruction unit 106 may determine the measured value as an abnormal value, and determine, as the point P3, a point closer to a point where the abnormal value is determined than the way point between the point P1 and the point P2. The instruction unit 106 may compare an average value of measured values which are measured in a same time slot by the sensor unit 30 at another point other than the point P1 and the point P2 with the measured value M1 and the measured value M2, and set, as the point P3, a point closer to the point P1 or the point P2, which has a larger difference from the average value than the other, than the way point between the point P1 and the point P2. The instruction unit 106 may decide a plurality of points P3 such that a number of points closer to the point P1 or the point P2 where the abnormal value is determined than the way point between the point P1 and the point P2 is increased as measurement points, and instruct the mobile robot 10 to perform the measurement at each of the points P3.

When a difference between the measured values in a same time slot of the measured value M1 or the measured value M2 is equal to or greater than a threshold, the determination unit 104 may determine that the mobile robot 10 is caused to measure the measured value M3 at the point P3 by the sensor unit 20. When the difference between the measured values in the same time slot of the measured value M1 or the measured value M2 is less than the threshold, the estimation unit 110 may estimate the measured value M3 at the point P3 by the interpolation between the measured value M1 and the measured value M2.

The estimation unit 110 may perform the interpolation by using a trained predictive model in which temperatures, humidities, and light quantities at the point P1 and the point P2 which are respectively measured by the sensor unit 30 (P1) and the sensor unit 30 (P2) are set as explanatory variables, and a temperature, a humidity, and a light quantity at the point P3 which are measured by the sensor unit 20 are set as objective variables.

By performing machine learning according to a supervised learning algorithm in which the temperatures, the humidities, and the light quantities at the point P1 and the point P2 which are respectively measured by the sensor unit 30 (P1) and the sensor unit 30 (P2) are set as the explanatory variables and the temperature, the humidity, and the light quantity at the point P3 which are measured by the sensor unit 20 are set as the objective variables, the generation unit 120 may generate a trained predictive model for predicting the temperature, the humidity, and the light quantity at the point P3 from the temperatures, the humidities, and the light quantities at the point P1 and the point P2, and store the trained model in the storage unit 130. The algorithm may be an algorithm of any method such as a neural network, a support vector machine, a multiple regression analysis, or a decision tree.

The generation unit 120 generates a measured value distribution of the indoor space of the plant factory based on the measured value M1, the measured value M2, and the measured value M3. The generation unit 120 generates the measured value distribution of the indoor space of the plant factory based on the measured value at each point measured by the sensor unit 20 and the sensor unit 30 and the measured value at each point estimated by the estimation unit 110. The measured value distribution may include at least one distribution of the temperature, the humidity (moisture content), the electrical conductivity, or the hydrogen ion index of the root part of the plant 50, or the front surface temperature, the ambient temperature, the humidity, the light quantity, or the carbon dioxide concentration of the stem, leave, and fruit part of the plant 50. The measured value distribution may include at least one distribution of the temperature, the humidity (moisture content), the electrical conductivity, or the hydrogen ion index of the root part of the plant 50, and at least one distribution of the front surface temperature, the ambient temperature, the humidity, the light quantity, or the carbon dioxide concentration of the stem, leave, and fruit part of the plant 50. The measured value distribution may include respective distributions of the temperature, the humidity, the electrical conductivity, and the hydrogen ion index of the root part of the plant 50, and respective distributions of the front surface temperature, the ambient temperature, the humidity, the light quantity, and the carbon dioxide concentration of the stem, leave, and fruit part of the plant 50.

The instruction unit 106 instructs at least one of the nutrient solution supply equipment 400, the light source equipment 410, the air handling equipment 420, the air blowing equipment 430, and the carbon dioxide supply equipment 440 in the farm field to adjust at least one of a moisture content, an electrical conductivity, or a hydrogen ion index of a soil in the farm field or a temperature, a humidity, a light quantity, or a carbon dioxide concentration in the farm field based on a predictive model representing a relationship between the measured value distribution of the indoor space of the plant factory and an occurrence status of a defect of the plant 50 or a harvest result of the plant 50 such that the plant 50 reaches a predetermined growing condition. The instruction unit 106 may instruct at least one of the nutrient solution supply equipment 400, the light source equipment 410, the air handling equipment 420, the air blowing equipment 430, and the carbon dioxide supply equipment 440 in the farm field to adjust at least one of the moisture content, the electrical conductivity, or the hydrogen ion index of the soil in the farm field or the temperature, the humidity, the light quantity, or the carbon dioxide concentration in the farm field based on a predictive model representing a relationship between the measured value distribution of the root part of the plant 50 and the measured value distribution of the stem, leave, and fruit part of the plant 50 and the occurrence status of the defect of the plant 50 or the harvest result of the plant 50 such that the plant 50 reaches the predetermined growing condition. The instruction unit 106 may identify the growing condition of the plant 50 based on three-dimensional position information of the plant 50 which is obtained by the detection result by the optical sensor such as the camera or the laser scanner which exists in the farm field. The sensor unit 20 included in the mobile robot 10 may have the optical sensor such as the camera or the laser scanner for generating the three-dimensional position information.

By performing the machine learning according to the supervised learning algorithm in which cultivation condition data representing each of the measured value distributions is set as as the explanatory variable and cultivation result data representing the occurrence status of the defect of the plant 50 or the harvest result of the plant 50 is set as the objective variable, the generation unit 120 may generate a trained predictive model which predicts the occurrence status of the defect of the plant 50 or the harvest result of the plant 50 from each of the measured value distributions, and store the trained model in the storage unit 130. The algorithm may be an algorithm of any method such as a neural network, a support vector machine, a multiple regression analysis, or a decision tree.

The cultivation result data may include at least one of the defect in the cultivation of the plant 50 or the harvest result of the plant 50. The defect in the cultivation of the plant 50 may include at least one of a physiological defect such as blossom-end rot and fruit cracking, a defect due to a disease, or a defect due to a pest. Data representing the defect of the plant 50 may include at least one of the presence or absence of the occurrence, a type of the defect, an occurrence frequency (for example, a percentage of the plant in which the defect occurs in cultivation units), or a range. The harvest result of the plant 50 may include at least one of a weight, a number, or a quality (a sugar content, a water content, or the like) of the harvested plant.

The generation unit 120 may have a preprocessing unit 122, a class estimation unit 124, a model generation unit 126, and a model update unit 128.

The preprocessing unit 122 performs preprocessing on at least one data of the plurality of pieces of cultivation condition data and the plurality of pieces of cultivation result data which are stored in the storage unit 130, and supplies the preprocessed data to the class estimation unit 124. The preprocessing unit 122 may perform the preprocessing for learning.

The class estimation unit 124 classifies a plurality of pieces of cultivation condition data into a plurality of classes. The class estimation unit 124 may supply the plurality of pieces of classified cultivation condition data to the model generation unit 126, the estimation unit 110, and the model update unit 128.

The model generation unit 126 generates a trained model which predicts cultivation result data from the cultivation condition data by using the cultivation condition data and the cultivation result data, and stores the trained model in the storage unit 130. The model update unit 128 updates the trained model by using the cultivation condition data and the cultivation result data.

The preprocessing unit 122 may associate the cultivation condition data and the cultivation result data according to an acquisition time period of the measured values from the sensor unit 20 and the sensor unit 30. As an example, the preprocessing unit 122 may associate the cultivation condition data and the cultivation result data according to the acquisition time period of the measured values on a same acquisition date basis or on a same time interval basis on a different acquisition date or a same acquisition date.

The preprocessing unit 122 may perform missing interpolation of data such as measured values in a time sequence. The preprocessing unit 122 may interpolate data by using an interpolation algorithm such as linear interpolation or spline interpolation with regard to a period during which data does not exist. When a difference of certain data among data from an average value of the plurality of pieces of data exceeds a threshold predetermined by a user, the preprocessing unit 122 may delete the data as an outlier or change the data to a same value as a value of data before or after on a timeframe. The preprocessing unit 122 may perform rounding processing on the data through truncation or the like of a predetermined digit and below. The preprocessing unit 122 may discretize the cultivation condition data and the cultivation result data in association with the cultivation area in each cultivation period (sowing, raising of seedlings, planting, and greening).

The preprocessing unit 122 may perform preprocessing of extracting feature amounts of the cultivation condition data and the cultivation result data. In at least one of the cultivation condition data or the cultivation result data, the preprocessing unit 122 may extract, as a feature amount, at least one of an integrated value, a differential value, an average value, a variance value, or data obtained by separating a daytime component and a nighttime component from each other. The preprocessing unit 122 may calculate, as the feature amount, the integrated value of data for each predetermined period. The preprocessing unit 122 may extract an integrated value of a number of occurrences of defects, a temperature, a humidity, or the like as a feature amount every three hours as an example. In this manner, effects that appear in a delayed manner can be found out by calculating the integrated value. The preprocessing unit 122 may calculate a differential value of the data during a plurality of data acquisition times, and extract the differential value as the feature amount. The preprocessing unit 122 may regard transitional data on the timeframe as a composite wave of a daytime component and a nighttime component, and separate one of the daytime component or the nighttime component by interpolation (for example, spline interpolation) or a frequency decomposition technique (for example, Fourier transform or the like). Then, the preprocessing unit 122 may separate a difference between the separated one of the daytime component or the nighttime component and the sensor data as the other of the daytime component or the nighttime component.

The preprocessing unit 122 may divide the preprocessed data in segments such as cultivation periods or time slots (the daytime and the nighttime), and create a data set by setting the preprocessed data (such as an average value or a variance for each segment) as the explanatory variable. The preprocessing unit 122 supplies a preprocessed data set to the class estimation unit 124.

The class estimation unit 124 classifies the plurality of preprocessed cultivation condition data into a plurality of classes (clustering). The class estimation unit 124 may classifies a plurality of identifiers of the plant 50 into a plurality of classes (groups) with a similar set of the cultivation condition data. The class estimation unit 124 may classify the data based on at least one of a time sequence or a similarity of a feature amount or the like with regard to the data. As an example, the class estimation unit 124 may classify, into the same class, mutual pieces of data in the same time slot on different acquisition dates. In addition, the class estimation unit 124 may classify, into the same class, mutual pieces of data in which a similarity in vectorized data is higher than a threshold value (for example, a distance is less than the threshold value). As an example, the class estimation unit 124 may classify, into the same class, data sets in which a difference between extracted feature amounts is lower than or equal to the threshold value and which correspond to each other.

The class estimation unit 124 may perform classification by using a k-means technique, a probabilistic latent semantic analysis (pLSA), or the like.

The class estimation unit 124 may create a class estimation model in which a result of the classification is set as the objective variable, and the preprocessed data set is set as the explanatory variable. The class estimation model may be created by using machine learning of a Bayesian network or the like. The class estimation unit 124 may estimate the class of the cultivation condition data by using the class estimation model in an output operation of the prediction result which will be performed later. In addition, the class estimation unit 124 may use information of the class used to generate the trained model in the output operation of the prediction result which will be performed later.

After the clustering, the class estimation unit 124 may supply a part of the cultivation condition data and a part of the corresponding cultivation result data to the model generation unit 126 as data for generating the model. After the clustering, the class estimation unit 124 may supply another part of the cultivation condition data and another part of the corresponding cultivation result data to the estimation unit 110 as data for calculating degree of confidence. The class estimation unit 124 may divide the data for generating the model and the data for calculating the degree of confidence to calculate the degree of confidence by using cross validation or the like. The class estimation unit 124 may randomly divide the data for generating the model and the data for calculating the degree of confidence. In addition, the class estimation unit 124 may divide the data for generating the model and the data for calculating the degree of confidence by a data acquisition period (for example, the daytime and the nighttime, a date, or a month).

The model generation unit 126 generates the trained model by performing machine learning by using the result of the classification (classified data). The model generation unit 126 may receive, from the class estimation unit 124, part of the cultivation condition data representing various types of measured values acquired by the acquisition unit 102 and the plurality of pieces of cultivation result data identified from the various types of measured values as data for generating the model, and generate the trained model by using the data for generating the model. The model generation unit 126 may generate a model of a Bayesian network model structure by using the plurality of pieces of cultivation condition data and the plurality of pieces of cultivation result data which are classified in to the plurality of classes. In addition, the model generation unit 126 may generate another machine learning model such as a neural network.

In addition, when the trained model is already stored in the storage unit 130, the model update unit 128 updates the trained model by using the result of the classification (classified data). The model update unit 128 may perform machine learning similarly as in the model generation unit 126 to update the trained model of the Bayesian network model structure. The model update unit 128 may compare a cultivation result under an executed cultivation condition of the plant 50 with a cultivation result predicted from the cultivation condition by the trained model to update the trained model.

The estimation unit 110 predicts at least one of the defect in the cultivation of the plant 50 or the cultivation result by using the trained model. The estimation unit 110 may receive the data for calculating the degree of confidence including the result of the classification (classified data) from the class estimation unit 124, and predict at least one of the defect or the cultivation result in the cultivation of the plant 50 from the data by using the trained model. The estimation unit 110 may predict one defect or cultivation result that has a highest probability or a probability above a threshold from the cultivation condition corresponding to one identifier by using the trained model.

The estimation unit 110 may calculate the degree of confidence in each of the plurality of classes by using the predicted defect or cultivation result and the data for calculating the degree of confidence which is used for the prediction. With regard to a class to which a cultivation condition used for the prediction is estimated to belong, the estimation unit 110 may compare the actual defect or cultivation result under the cultivation condition with the predicted defect or cultivation result to calculate the degree of confidence (accuracy rate). The estimation unit 110 may calculate the degree of confidence for each identifier, and calculate one final degree of confidence from the plurality of degrees of confidence for each class. With regard to one class, the estimation unit 110 may calculate an average value or total value of the plurality of degrees of confidence calculated with regard to the plurality of identifiers as the final degree of confidence of the class. The estimation unit 110 may calculate the degree of confidence by using at least one of a recall rate, a precision rate, or an F value.

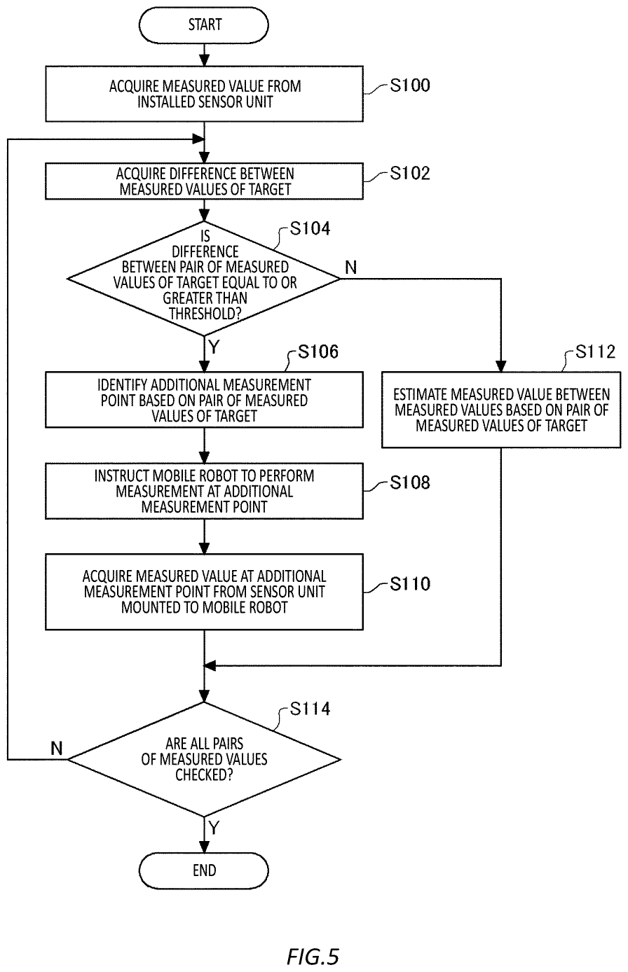

FIG. 5 is a flowchart illustrating an example of a procedure to determine whether a measured value between points is acquired by an estimation or is acquired by a measurement by using the mobile robot 10.

The acquisition unit 102 acquires measured values detected by various types of sensors from the sensor units 30 installed at respective points of the cultivation rack 60 (S100). The determination unit 104 acquires a difference between the measured values of the target (S102). Information indicating a pair of the measured values the difference of which is derived by the determination unit 104, that is, a pair of the points, may be stored in the storage unit 130 in advance. The pair of the points may be a pair of the sensor units 30 next to each other. The pair of the points may be a pair of the sensor units 30 next to each other along a longitudinal direction of the cultivation rack 60. The pair of the points may be a pair of points at a shortest distance among pairs of points where at least one plant 50 that is not set as a measurement target by the sensor unit 30 exists therebetween.

When a difference of the pair of the measured values of the target is equal to or greater than a threshold (“Y” in S104), the instruction unit 106 identifies at least one additional measurement point based on the pair of the measured values of the target (S106). The instruction unit 106 may identify a midpoint between the points of the pair of the measured values of the target as the additional measurement point. The instruction unit 106 may identify the additional measurement point based on each value of the pair of the measured values of the target. The instruction unit 106 may identify, as the additional measurement point, a point closer to the point with the measured value having a larger difference with respect to an average measured value at a same point in a same time slot in the past out of the pair of the measured values of the target. The instruction unit 106 may identify a plurality of additional measurement points set to be equally spaced between the points of the pair of the measured values of the target.

The instruction unit 106 instructs the mobile robot 10 to perform the measurement at the identified additional measurement point (S106). In the storage unit 130, respective planting pots 62 of the cultivation rack 60 and numbers for uniquely identifying the respective planting pots 62 may be stored in association with each other. The mobile robot 10 may hold map information indicating a position of the planting pot 62 corresponding to the number in the memory. The instruction unit 106 may instruct the mobile robot 10 to perform the measurement at the additional measurement point by outputting a measurement instruction command indicating the number corresponding to the additional measurement point to the mobile robot 10.

In response to the instruction, the mobile robot 10 may move to the additional measurement point, and the sensor unit 20 may measure the temperature, the humidity, the electrical conductivity, and the hydrogen ion index of the root part of the plant 50 of the target, and the temperature, the humidity, the light quantity, and the carbon dioxide concentration of the stem, leave, and fruit part of the plant 50. The mobile robot 10 may perform respective measurements by various types of sensors included in the sensor unit 20 by controlling the arm 12 while identifying the position of the plant 50 by the camera provided in the arm 12. The acquisition unit 102 acquires the measured value at the additional measurement points of various types of sensors from the sensor unit 20 (S110).

When the difference of the pair of the measured values of the target is less than the threshold (“N” in S104), the estimation unit 110 estimates an measured value at the additional measurement point between the measured values by interpolation based on the pair of the measured values of the target (S112).

The determination unit 104 determines whether the measurement or the estimation of additional measured values is performed with regard to all the pairs of the measured values measured in a same time slot (S114), and when the determination with regard to all the pairs of the measured values is not performed, the determination unit 104 repeats the processing in step S102 and subsequent steps.

As described above, in accordance with the farm field management apparatus 100 according to the present embodiment, when the difference between the measured values is large, the mobile robot 10 moves to another measurement point between the measurement points and actually performs the measurement at the point by the sensor unit 20. On the other hand, when the difference between the measured values is small, instead of the measurement by the mobile robot 10, the measured value at another measurement point between the measurement points is estimated by interpolation. Thus, while a number of sensor units provided in the farm field is suppressed, it is possible to suppress the fall of the precision in the grasp of the environmental condition in the surrounding of the individual plant 50 and the growing condition of the plant 50.

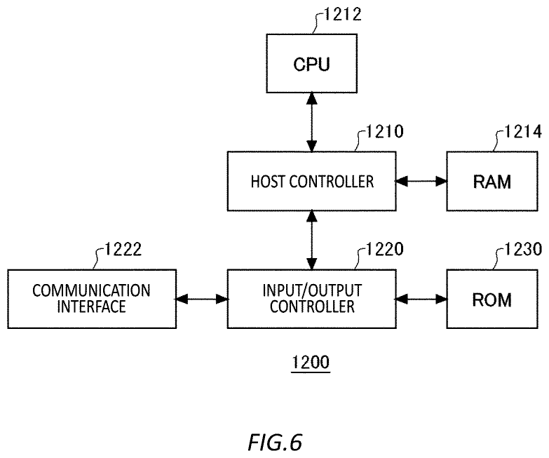

FIG. 6 illustrates an example of a computer 1200 in which aspects of the present embodiment may be entirely or partially embodied. Programs installed in the computer 1200 can cause the computer 1200 to function as operations associated with the apparatus according to the embodiments of the present invention or one or more “units” of the apparatuses. Alternatively, the programs can cause the computer 1200 to execute the operations or the one or more “units”. The programs can cause the computer 1200 to execute a process according to the embodiments of the present invention or steps of the process. Such programs may be executed by a CPU 1212 to cause the computer 1200 to perform specific operations associated with some or all of the blocks in the flowcharts and block diagrams described in the present specification.

The computer 1200 according to the present embodiment includes the CPU 1212 and a RAM 1214, which are mutually connected by a host controller 1210. The computer 1200 also includes a communication interface 1222 and an input/output unit, which are connected to the host controller 1210 via an input/output controller 1220. The computer 1200 also includes a ROM 1230. The CPU 1212 operates according to the programs stored in the ROM 1230 and the RAM 1214, thereby controlling each unit.

The communication interface 1222 communicates with other electronic devices via a network. A hard disk drive may store the programs and data used by the CPU 1212 in the computer 1200. The ROM 1230 stores therein boot programs or the like executed by the computer 1200 at the time of activation, and/or stores programs depending on hardware of the computer 1200. The programs are provided via a computer readable storage medium such as CR-ROM, a USB memory or an IC Card or a network. The programs are installed on the RAM 1214, which also is an example of the computer readable storage medium, or the ROM 1230 and performed by the CPU 1212. Information processing written in these programs is read by the computer 1200, and provides cooperation between the programs and the various types of hardware resources described above. An apparatus or a method may be configured by implementing operations or processing of information according to a use of the computer 1200.

For example, in a case where a communication is performed between the computer 1200 and an external device, the CPU 1212 may execute a communication program loaded in the RAM 1214 and instruct the communication interface 1222 to perform communication processing based on a process written in the communication program. Under the control of the CPU 1212, the communication interface 1222 reads transmission data which is stored in the RAM 1214 or a transmission buffer region which is provided in a storage media such as a USB memory, to transmit the read transmission data to the network or write the reception data received from the network into a reception buffer region or the like provided on the storage media.

Also, the CPU 1212 may cause the whole or required part of files which are stored in the external storage media (such as USB memory) or the database to be read by the RAM 1214, to perform a various type of processes for the data on the RAM 1214. Then, the CPU 1212 may write back the processed data to the external storage media.

A various type of information such as a various type of programs, data, tables and databases may be stored in a storage media to undergo an information processing. The CPU 1212 may execute, on the data read from the RAM 1214, various types of processing including various types of operations, information processing, conditional judgement, conditional branching, unconditional branching, information retrieval/replacement, or the like described throughout the present disclosure and specified by instruction sequences of the programs, to write the results back to the RAM 1214. Also, the CPU 1212 may retrieve information in the file, database or the like in the storage media. For example, when a plurality of entries each having an attribute value of the first attribute associated with an attribute value of the second attribute are stored in a storage media, the CPU 1212 may retrieve, among the plurality of entries, an entry whose attribute value of the first attribute is specified and matches the conditions and read the attribute value of the second attribute stored in the entry, thereby acquiring the attribute value of the second attribute associated with the first attribute which satisfies a predetermined condition.

The programs or software module described above may be stored on the computer 1200 or in a computer readable storage medium near the computer 1200. In addition, a storage medium such as a hard disk or a RAM provided in a server system connected to a dedicated communication network or the Internet can be used as the computer readable storage medium, thereby providing the program to the computer 1200 via the network.

A computer readable medium may include any tangible device that can store instructions to be executed by a suitable device. As a result, the computer readable medium having instructions stored therein includes an article of manufacture including instructions which can be executed in order to create means for performing operations specified in the flowcharts or block diagrams. Examples of the computer readable medium may include an electronic storage medium, a magnetic storage medium, an optical storage medium, an electromagnetic storage medium, a semiconductor storage medium, and the like. More specific examples of the computer readable medium may include a floppy disk, a diskette, a hard disk, a random access memory (RAM), a read only memory (ROM), an erasable programmable read only memory (EPROM or a flash memory), an electrically erasable programmable read only memory (EEPROM (registered trademark)), a static random access memory (SRAM), a compact disc read only memory (CD-ROM), a digital versatile disk (DVD), a Blu-ray (registered trademark) disk, a memory stick, an integrated circuit card, and the like.

A computer readable instruction may include either a source code or an object code described in any combination of one or more programming languages. The source code or the object code includes a conventional procedural programming language. The conventional procedural programming language may be assembler instructions, instruction-set-architecture (ISA) instructions, machine instructions, machine dependent instructions, microcode, firmware instructions, state-setting data, or an object oriented programming language such as Smalltalk (registered trademark), JAVA (registered trademark), C++, etc., and programming languages, such as the “C” programming language or similar programming languages. Computer readable instructions may be provided to a processor of a general purpose computer, special purpose computer, or other programmable data processing device, or to programmable circuitry, locally or via a local area network (LAN), a wide area network (WAN) such as the Internet, etc. The processor or the programmable circuitry may execute the computer readable instructions in order to create means for performing operations specified in the flowcharts or block diagrams. An example of the processor includes a computer processor, processing unit, microprocessor, digital signal processor, controller, microcontroller, or the like.

While the present invention has been described above by way of the embodiments, the technical scope of the present invention is not limited to the above described embodiments. It is apparent to persons skilled in the art that various alterations or improvements can be made to the above described embodiments. It is apparent from the description of the claims that embodiments added with such alterations or improvements can also be included in the technical scope of the present invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### EXPLANATION OF REFERENCES

10: mobile robot;
12: arm;
20, 30: sensor unit;
30: sensor unit;
50: plant;
60: cultivation rack;
62: planting pot;
80: network;
100: farm field management apparatus;
102: acquisition unit;
104: determination unit;
106: instruction unit;
110: estimation unit;
120: generation unit;
122: preprocessing unit;
124: class estimation unit;
126: model generation unit;
128: model update unit;
130: storage unit;
300: sensor management apparatus;
400: nutrient solution supply equipment;
410: light source equipment;
420: air handling equipment;
430: air blowing equipment;
440: carbon dioxide supply equipment;
1200: computer;
1210: host controller;
1212: CPU;
1214: RAM;
1220: input/output controller;
1222: communication interface;
1230: ROM.

1. A farm field management apparatus comprising a processor, wherein the processor:
acquires a first measured value at a first point in a farm field which is measured by a first sensor provided at the first point, and a second measured value at a second point in the farm field which is measured by a second sensor provided at the second point;
determines whether to cause a mobile robot to which a third sensor is mounted to move to a third point between the first point and the second point based on the first measured value and the second measured value and to cause the mobile robot to measure a third measured value at the third point by the third sensor; and
instructs, when the determination unit determines that the third measured value at the third point is to be measured, the mobile robot to perform measurement at the third point.

2. The farm field management apparatus according to claim 1, wherein when a difference between the first measured value and the second measured value is equal to or greater than a threshold, the processor determines that the mobile robot is to be caused to measure the third measured value at the third point by the third sensor.

3. The farm field management apparatus according to claim 1, wherein the processor estimates, when a difference between the first measured value and the second measured value is less than a threshold, the third measured value at the third point by interpolation between the first measured value and the second measured value.

4. The farm field management apparatus according to claim 1, wherein when a difference between measured values in a same time slot of the first measured value or the second measured value is equal to or greater than a threshold, the processor determines that the mobile robot is to be caused to measure the third measured value at the third point by the third sensor.

5. The farm field management apparatus according to claim 1, wherein the processor estimates, when a difference between measured values in a same time slot of the first measured value or the second measured value is less than a threshold, the third measured value at the third point by interpolation between the first measured value and the second measured value.

6. The farm field management apparatus according to claim 3, wherein the processor performs the interpolation by using a trained predictive model in which temperatures, humidities, and light quantities at the first point and the second point which are respectively measured by the first sensor and the second sensor are set as explanatory variables, and a temperature, a humidity, and a light quantity at the third point which are measured by the third sensor unit are set as objective variables.

7. The farm field management apparatus according to claim 2, wherein the processor instructs the mobile robot to perform the measurement at the third point in a manner that as the difference between the first measured value and the second measured value becomes larger, a frequency to measure the third measured value at the third point by the third sensor is increased.

8. The farm field management apparatus according to claim 1, wherein the third point includes a plurality of third points, and when a difference between the first measured value and the second measured value is equal to or greater than a threshold, the processor determines that the mobile robot is to be caused to measure the third measured value at each of the plurality of third points by the third sensor.

9. The farm field management apparatus according to claim 8, wherein the processor instructs the mobile robot to perform the measurement at the third point in a manner that as the difference between the first measured value and the second measured value becomes larger, a number of the third points at which the mobile robot is instructed to perform the measurement is increased.

10. The farm field management apparatus according to claim 1, wherein the processor decides a position of the third point based on the first measured value and the second measured value.

11. The farm field management apparatus according to claim 1, wherein the processor generates a measured value distribution of the farm field based on the first measured value, the second measured value, and the third measured value.

12. The farm field management apparatus according to claim 11, wherein the measured value distribution includes at least one distribution of a temperature, a humidity, an electrical conductivity, or a hydrogen ion index of a root part of a plant, or a front surface temperature, an ambient temperature, a humidity, a light quantity, or a carbon dioxide concentration of a stem, leave, and fruit part of the plant.

13. The farm field management apparatus according to claim 12, wherein the processor instructs environmental control equipment in the farm field to adjust at least one of a moisture content, an electrical conductivity, or a hydrogen ion index of a soil in the farm field or a temperature, a humidity, a light quantity, or a carbon dioxide concentration in the farm field based on a predictive model representing a relationship between the measured value distribution and an occurrence status of a defect of the plant or a harvest result of the plant such that the plant reaches a predetermined growing condition.

14. The farm field management apparatus according to claim 13, wherein the processor identifies the growing condition of the plant based on three-dimensional position information of the plant which is obtained by a detection result by an optical sensor existing in the farm field.

15. The farm field management apparatus according to claim 14, wherein the optical sensor is mounted to the mobile robot.

16. A farm field management method comprising:
acquiring a first measured value at a first point in a farm field which is measured by a first sensor provided at the first point, and a second measured value at a second point in the farm field which is measured by a second sensor provided at the second point;
determining whether to cause a mobile robot to which a third sensor is mounted to move to a third point between the first point and the second point based on the first measured value and the second measured value and to cause the mobile robot to measure a third measured value at the third point by the third sensor; and
instructing, when it is determined in the determining that the third measured value at the third point is to be measured, the mobile robot to perform measurement at the third point.

17. A non-transitory computer readable medium having recorded thereon a program for, when executed by a computer, causing the computer to perform:
acquiring a first measured value at a first point in a farm field which is measured by a first sensor provided at the first point, and a second measured value at a second point in the farm field which is measured by a second sensor provided at the second point;
determining whether to cause a mobile robot to which a third sensor is mounted to move to a third point between the first point and the second point based on the first measured value and the second measured value and to cause the mobile robot to measure a third measured value at the third point by the third sensor; and
instructing, when the computer determines that the third measured value at the third point is to be measured, the mobile robot to perform measurement at the third point.

18. The farm field management apparatus according to claim 2, wherein the processor estimates, when a difference between measured values in a same time slot of the first measured value or the second measured value is less than a threshold, the third measured value at the third point by interpolation between the first measured value and the second measured value.

19. The farm field management apparatus according to claim 5, wherein the processor performs the interpolation by using a trained predictive model in which temperatures, humidities, and light quantities at the first point and the second point which are respectively measured by the first sensor and the second sensor are set as explanatory variables, and a temperature, a humidity, and a light quantity at the third point which are measured by the third sensor unit are set as objective variables.

20. The farm field management apparatus according to claim 2, wherein the processor generates a measured value distribution of the farm field based on the first measured value, the second measured value, and the third measured value.
